(19) United States
(12) Patent Application Publication
(10) Pub. No.: US 2023/0233477 A1
(43) Pub. Date: Jul. 27, 2023

(54) Bi2O3 NANOPARTICLES PREPARED BY THE TOP-DOWN ULTRASONICATION ROUTE AS A BROAD-SPECTRUM ANTIMICROBIAL TO OVERCOME DRUG RESISTANCE IN ANTIBIOTICS

(71) Applicant: KENT STATE UNIVERSITY, Kent, OH (US)

(72) Inventors: Songping D. HUANG, Kent, OH (US); Min-Ho Kim, Hudson, OH (US); Bishnu D. PANT, Kent, OH (US); Bogdan M. Benin, Twinsburg, OH (US); K.G. Nalin Krishantha ABEYdeera, Kent, OH (US)

(21) Appl. No.: 18/102,272

(22) Filed: Jan. 27, 2023

Related U.S. Application Data
(60) Provisional application No. 63/303,644, filed on Jan. 27, 2022.

(51) Int. Cl. A61K 9/51; A61K 33/245

(52) U.S. Cl. CPC A61K 9/5192; A61K 33/245; A61K 9/5146; A61K 9/5138

(57) ABSTRACT

α-Bi₂O₃ NPs exhibit not only potent broad-spectrum antibacterial activity of killing both Gram-negative (MIC=0.75 μg/mL vs. P. aeruginosa) and Gram-positive (MIC=2.5 μg/mL vs. S. aureus) bacteria, but they are also effective against Ag-resistant and carbapenem-resistant bacteria (MICs=1.0 μg/mL and 1.25 μg/mL, respectively), and they are able to sensitize bacteria towards meropenem (mero), acting synergistically and thus allowing for its continued use with smaller therapeutic doses (fractional inhibitory concentration=0.45). Importantly, unlike other technologies that have been considered as effective metal antimicrobials, α-Bi₂O₃ NPs do not contribute to the generation of antimicrobial resistant phenotypes with no resistance observed after 30 passages. The Bi-based materials represent a critical tool against multidrug resistant bacteria.

### FIELD OF THE INVENTION

Antimicrobial resistance is an ongoing and increasing threat to global public health. In order to combat the spread of pathogenic bacteria, numerous antimicrobial materials have been investigated and incorporated into wound dressings and medical devices such as implants and catheters. The most frequently utilized of these materials are Ag salts and Ag nanoparticles (NPs) due to their impressively low minimum inhibitory concentrations (MICs) against common Gram-negative pathogenic bacteria such as P. aeruginosa. However, Ag-based compounds and AgNPs are limited to treating Gram-negative bacteria, and they have been demonstrated to generate Ag-resistant phenotypes after only 7 days of consecutive exposure to a sub-inhibitory concentration. Here we demonstrate that novel, polymer-coated α-Bi₂O₃ NPs exhibit not only potent broad-spectrum antibacterial activity of killing both Gram-negative (MIC=0.75 μg/mL vs. P. aeruginosa) and Gram-positive (MIC=2.5 μg/mL vs. S. aureus) bacteria, but they are also effective against Ag-resistant and carbapenem-resistant bacteria (MICs=1.0 μg/mL and 1.25 μg/mL, respectively), and that they are able to sensitize bacteria towards meropenem (mero), acting synergistically and thus allowing for its continued use with smaller therapeutic doses (fractional inhibitory concentration=0.45). Importantly, unlike other technologies that have been considered as effective metal antimicrobials, α-Bi₂O₃ NPs do not contribute to the generation of antimicrobial resistant phenotypes with no resistance observed after 30 passages. Our results demonstrate that Bi-based materials represent a critical tool against multidrug resistant bacteria and require renewed and greater attention within the community.

### BACKGROUND OF THE INVENTION

The World Health Organization has identified antimicrobial resistance (AMR) as one of the greatest threats to global health and development.¹ The worldwide increase in AMR is directly tied to the widespread use of antibiotics in both human and veterinary medicine, with infections caused by multidrug resistant (MDR) bacteria resulting in significant increases to health care costs ($4.6 billion in 2017) while also leading to increased morbidity and mortality for patients.² Although numerous antibiotic stewardship and monitoring programs have been initiated around the world, the lack of next generation antibiotics that are in development, globally, remains a critical need in the effort to confront AMR.³

Current antibiotics are typically organic molecules comprising various classes such as fluoroquinolones, cephalosporins, penicillins, etc. These tend to have specific intracellular targets such as cell wall synthesis or topoisomerase IV; however, extensive use of these agents has resulted in the generation of resistant phenotypes with various mutations reducing the efficacy of such antibiotics.^{4,5}

One alternative strategy centers on the use of antimicrobial metals and metallic nanoparticles. Such materials have long been known to be bactericidal at low concentrations,⁶ with this effect being generally observed in metals and alloys such as copper, silver, and bronze as early as ancient times.⁷ Of these materials, silver is considered one of the most potent, especially when prepared as AgNPs.⁸ While these typically have MIC values of 1 μg/mL to 5 μg/mL against Gram-negative bacteria, depending on size and morphology,^{9,10} AgNPs are known to be less effective against Gram-positive bacteria including Staphylococcus aureus with a reported MIC value>1800 μg/mL for 10 nm AgNPs.¹¹ Even though AgNPs are impressively effective in killing Gram-negative bacteria, it was recently reported that such NPs also resulted in the growth of resistant phenotypes,^{9,12} thus calling into question the continuous and long-term utility of these nanomaterials in various formulations, wound dressings, medical devices, or other common household items.¹³⁻¹⁵ Additionally, AgNPs released to the environment are highly toxic to aquatic life forms as well as harming benign bacteria and microorganisms beneficial to such ecosystems.^{16,17} These shortcomings of Ag-based nanomaterials, necessitate the pursuit of alternative antimicrobial nanomaterials given both the potency and the possibility of nanomaterials to exhibit significantly decreased resistance development, owing to their alternative antimicrobial mechanisms of action as compared to those of conventional antibiotics.

To address this need, we have investigated the use of Bi-based nanomaterials since Bi has been utilized both historically and in modern medicine to treat a broad range of infections and gastrointestinal disorders.^{18,19} Additionally, Bi-based therapeutics or preparations tend to exhibit fewer side effects compared to the other metal-based counterparts and are reported to be well tolerated by patients due to their low toxicity.²⁰ In fact, previous studies have demonstrated the antimicrobial effects of Bi-based materials and prominent examples of commercial products exist such as Xeroform, a widely used wound dressing, including antimicrobial agents based on Bi.²¹⁻²⁵ Given these observations, we considered the use of Bi-based NPs as a potential antimicrobial agent, analogous to AgNPs. Unlike Ag, however, which has a relatively low affinity for oxygen and a moderate melting point of 961.8° C., Bi readily forms oxides and chalcogenides and cannot be easily prepared or delivered as metallic nanoparticles due to its very low bulk melting point (271.5° C.). We therefore considered Bi₂O₃ as the closest analogue to metallic Ag as a potential antimicrobial material (FIG. 1a). In support of this idea, several articles have previously reported the broad antibacterial activity of Bi₂O₃ nanomaterials, with MIC values typically on par with those of Ag NPs.^{26,27} Despite these promising reports, little is known about the details of this antibacterial activity or of the ability for Bi₂O₃ NPs to generate resistant bacterial phenotypes.

### SUMMARY OF THE INVENTION

Herein we report the exceptionally potent antimicrobial activity of compositions including polymer-coated Bi₂O₃ NPs against both Gram-negative and Gram-positive bacteria, their previously unreported superior resistance profiles as compared to other therapeutic metals, their activity against multi-drug resistant, silver resistant, and carbapenem resistant bacteria, as well as their growth inhibitory effect against biofilms and their ability to sensitize bacteria towards meropenem.

### BRIEF DESCRIPTION OF THE DRAWINGS

The invention will be better understood, and other features and advantages will become apparent by reading the detailed description of the invention, taken together with the drawings, wherein:

FIG. 1 relates to the characterization of Bi₂O₃ NPs, wherein a) unit cell of α-Bi₂O₃; b) illustrates experimental and simulated (α-Bi₂O₃) powder XRD patterns; c) is a TEM image of Bi₂O₃ NPs (inset: size distribution histogram); and d) is a HRTEM image of a single Bi₂O₃ NP;

FIG. 2 relates to antibacterial activity studies, wherein a) is a CFU analysis of Bi₂O₃ NPs for PA; b) illustrates time-kill curves of Bi₂O₃ NPs against PA with varying concentrations during 24-h incubation (mean±s.d, n=3 replicates); c) illustrates drug resistance development of Bi₂O₃ nanoparticles with comparison of Ciprofloxacin; and d) illustrates declined resistance from Bi₂O₃ nanoparticles treatment for samples which already developed drug resistance from Ag nanoparticles, ciprofloxacin and meropenem;

FIG. 3. relates to a mechanistic study of the antimicrobial activity of Bi₂O₃ NPs, wherein a) illustrates a cellular uptake of Bi₂O₃ NPs in PA bacteria as determined by the Bi concentration in the cell lysate after 6 hrs. of incubation. Normalized ROS generation analysis by varying concentrations of Bi₂O₃ NPs towards PA incubated for; b) 30 min; c) 45 min; and d) 60 min. Scanning electron microscope (SEM) images of PA wherein e) without Bi₂O₃ NPs, and after incubation with Bi₂O₃ NPs for; f) 30 min, g) 45 min; and h) 60 min. n.s. (not significant), *(p<0.05), **(p<0.01), ****(p<0.001), and ****(p<0.0001);

FIG. 4. illustrates a) growth-inhibitory effect of Bi₂O₃ nanoparticles with the corresponding concentrations on biofilm-derived MRSA bacterial cells; and b) illustrates checkerboard assay for Bi₂O₃ NPs and antibiotic Meropenem against DRPA. **(p<0.01), ****(p<0.001), and ****(p<0.0001);

FIG. 5A relates to Rietveld refinement of bulk Bi₂O₃. Reference patter calculated from ICSD #28443;

FIG. 5B relates to representative images of MIC test of Bi₂O₃-NPs, against (a) SA and (b) PA;

FIG. 5C relates to representative plot of CFU enumeration results of Bi₂O₃-NPs, against PA showing LD₅₀=0.5 ug/mL;

FIG. 5D relates to CFU analysis of Bi₂O₃ NPs for SA;

FIG. 5E relates to cell viability curves of Bi₂O₃ NPs against HDFs (a), RAW 264.7 cells and human fibroblast cells. (not significant), *(p<0.05), **(p<0.01), ****(p<0.001), and ****(p<0.0001) and

FIG. 6 illustrates the progressive increase in the zone of inhibition of PEG-based ointments containing 1%, 2% and 4% by weight of Bi₂O₃.

### DETAILED DESCRIPTION OF THE INVENTION

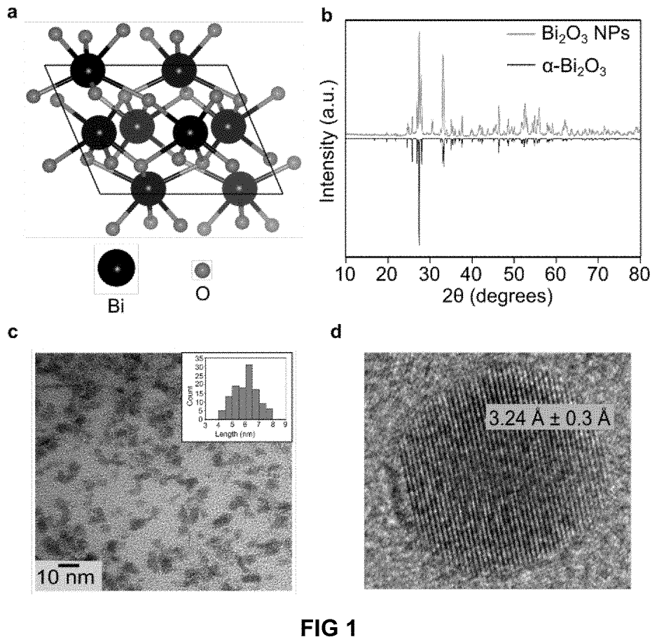

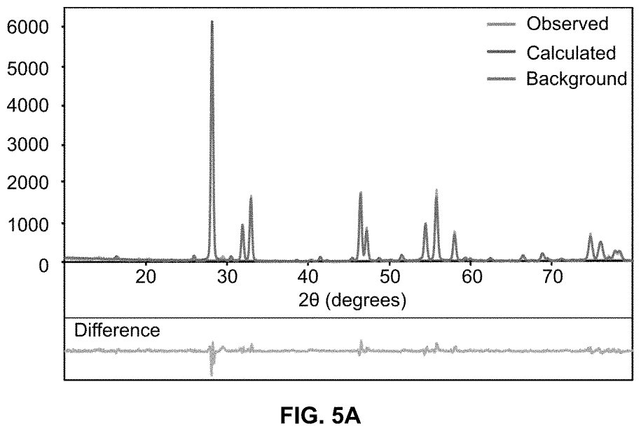

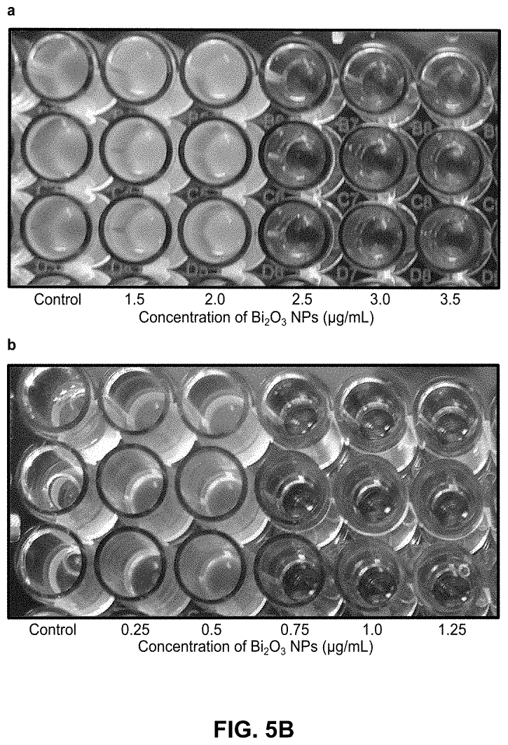

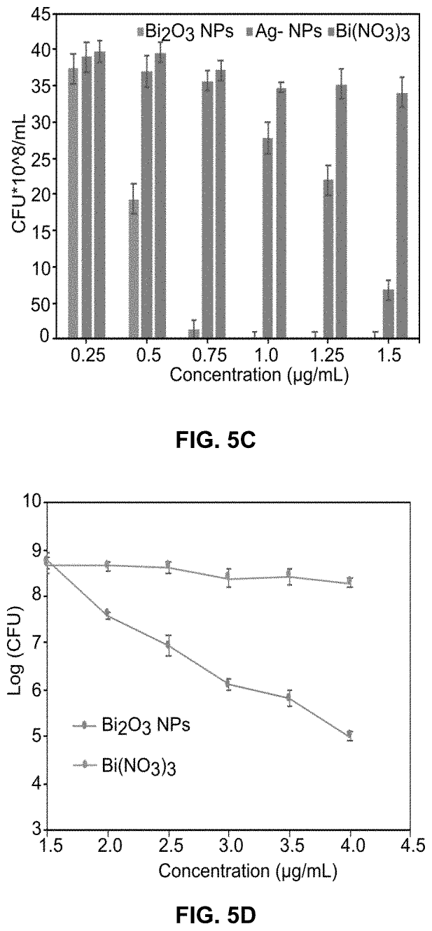

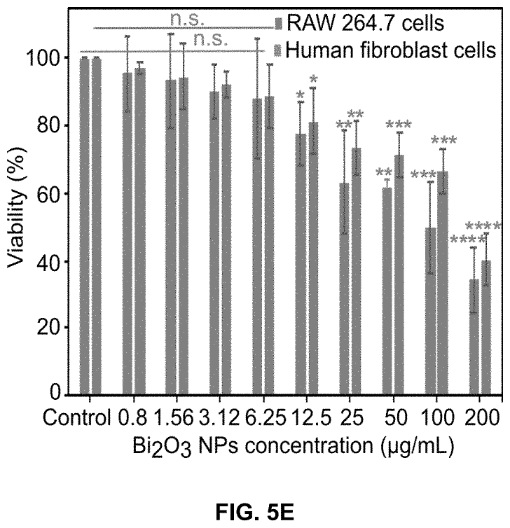

Bi₂O₃ NPs were obtained using a top-down sonomechanical approach in which bulk Bi₂O₃ was first synthesized following a standard solid-state sol-gel approach (FIG. 5A). This phase-pure bulk powder was then sonicated for 18 hrs. in the presence of polyvinylpyrrolidone (MW_{avg}=8000 g/mole, PVP-8k) and dimethyl sulfoxide (DMSO), yielding the final novel polymer-coated α-Bi₂O₃ NPs with some peak broadening, suggesting the presence of NPs (FIG. 1b).

The average size of the spherical NPs was determined to generally be from about 2 to about 50 nm, desirably from about 4 to about 30 nm, and preferably from about 6.03±0.93 nm using transmission electron microscopy (TEM), (FIG. 1c). High-resolution TEM (HRTEM) was used to further corroborate the phase present in the TEM image with the expected α-Bi₂O₃ phase by measuring the observed lattice spacing (FIG. 1d). Indeed, the observed lattice spacing of 3.24 Å±0.3 Å agrees well with the d-spacing (3.25 Å) of the highest intensity (120) peak of the PXRD pattern lattice plane.

Suitable solvents other than DMSO can be utilized, for example water, methanol, ethanol, N,N′-dimethyformamide (DMF) and acetonitrile. Still further, the Bi₂O₃ can be coated with water-soluble and biocompatible polymers other than PVP. Examples of suitable polymers include, but are not limited to polyethylene glycol, polyacrylamide, polyacrylic acid and polyvinyl alcohol.

Sonication is performed for a suitable period of time in order to reduce the particle size and coat the Bi₂O₃ with the polymer. Sonication time will vary depending upon the polymer and solvent utilized. That said, sonication generally takes place for a period of time ranging from about 1 hour to about 78 hours, and preferably from about 8 hours to about 24 hours. The process usually takes place at room temperature. Suitable temperatures range generally from 0° C. to 100° C., and the preferred temperature range is from 10° C. to 40° C.

Having successfully synthesized these NPs, we proceeded to first evaluate their antibacterial activity against Pseudomonas aeruginosa (PA)—a member of the ESKAPE group of pathogens that can develop and transfer carbapenamase genes.²⁸⁻³⁰ PA and multidrug resistant PA are both susceptible to Ag-based materials and AgNPs, providing a reference by which the activity of Bi₂O₃ NPs could be evaluated. After incubating PA (ATCC 15692) and DRPA (ATCC BAA 2108) with Bi₂O₃ NPs for 18 hrs., the MIC was determined to be 0.75 μg/mL (FIG. 5B). In comparison, AgNPs, which are considered to be one of the most potent antimicrobial materials, were previously reported to exhibit an MIC of 1.69 μg/mL vs. DRPA.

Given the obvious improvement in antibacterial activity of Bi₂O₃ NPs over AgNPs, we proceeded to evaluate whether Bi₂O₃ NPs may also be effective against Staphylococcus aureus (SA, ATCC 6538) and methicillin-resistant SA (MRSA, ATCC BAA 44) since AgNPs are known to be ineffective against Gram-positive bacteria and previous studies on Bi₂O₃ NPs were somewhat inconclusive.^{18,26} Impressively, MIC values of 2.5 μg/mL (FIG. S2) were observed for Bi₂O₃ NPs against both SA and MRSA, indicating that Bi₂O₃ NPs exhibit broad-spectrum antibacterial activity with much greater efficacy than AgNPs.

TABLE 1
MIC of Bi₂O₃ NPs against drug-sensitive
and drug-resistant bacterial strains.

| Species | Strain | MIC (μg/ml) |
|---|---|---|
| PA | ATCC 15692 | 0.75 |
| DRPA | ATCC BAA 2108 | 0.75 |
| SA | ATCC 6538 | 2.5 |
| MRSA | ATCC BAA 44 | 2.5 |

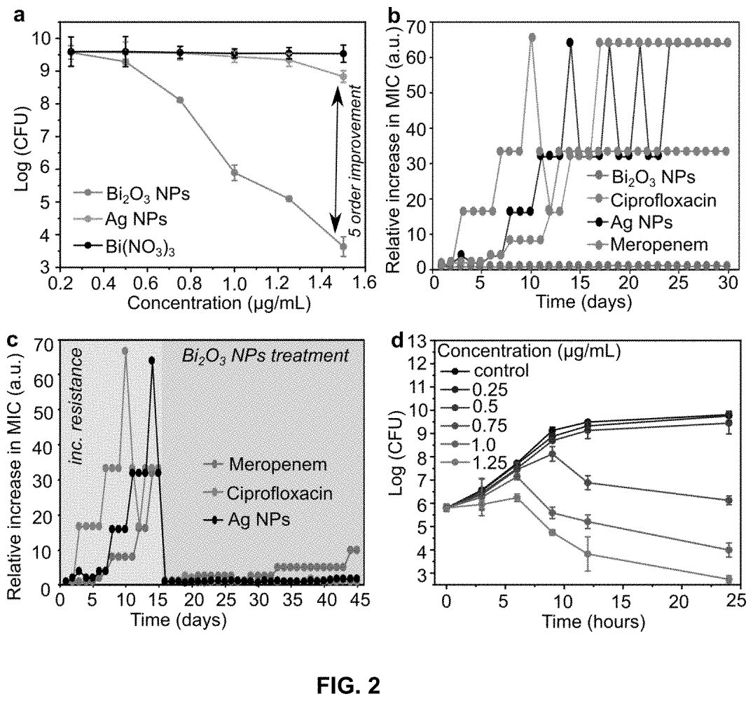

This improvement in antibacterial activity between Bi₂O₃ NPs and AgNPs was further demonstrated by colony forming unit (CFU) analysis. In this experiment, Log(CFU) reduction of PA was measured with respect to various concentrations of Bi₂O₃ NPs, AgNPs, and Bi(NO₃)₃ as a control. Of these, only Bi₂O₃ NPs demonstrated dose-dependent activity, with a half-maximal lethal dose (LD₅₀) of 0.5 μg/mL (FIG. 2a, FIG. 5C). Additionally, at a concentration of 1.5 μg/mL, Bi₂O₃ NPs demonstrated a 6-log reduction, which corresponds to complete eradication of the bacteria. AgNPs, on the other hand, only exhibited a modest 1-log reduction, in agreement with published MIC values. Furthermore, we obtained similar results when we treated SA with Bi₂O₃ NPs, emphasizing the broad-spectrum activity of these NPs (FIG. 5D).

Having demonstrated that Bi₂O₃ NPs are more effective antimicrobial agents than AgNPs, we sought to determine whether Bi₂O₃ NPs would also generate resistance as readily as AgNPs and whether they would be effective against Ag-resistant mutants.

First, cultures of PA were treated, separately, with Bi₂O₃ NPs, ciprofloxacin and Ag NPs as references, at a sub-lethal concentration for 30 successive passages (FIG. 2b). In the case of ciprofloxacin, the MIC increased 32-fold already after 13 days, indicating that PA was now resistant to ciprofloxacin. Similarly, AgNPs were no longer effective after 12 days (MIC=54 μg/mL, 32×MIC₀), and the MIC further increased to 64×MIC₀ after another 3 days, suggesting complete resistance by PA against AgNPs. Bi₂O₃ NPs, on the other hand, retained their initial MIC (0.75 μg/mL) throughout all 30-day passages, demonstrating that Bi₂O₃ NPs do not contribute to the additional generation of AMR.

Given the impressive ability of Bi₂O₃ NPs to avoid the generation of resistant phenotypes of PA, we considered whether these NPs would be able to kill PA that already exhibit varying types of resistance to AgNPs, ciprofloxacin, and meropenem (a last-line antibiotic in the carbapenem family; FIG. 2c).

To examine this, we first developed drug-resistant mutants of PA, which will be referred to as PA_{Ag}, PA_{cip}, and PAₘₑᵣₒ depending on whether they were developed with AgNPs, ciprofloxacin, or meropenem, respectively. PA mutants were considered to be resistant once their associated treatment exhibited a minimum of 32×MIC₀. All mutants were taken at day 15 when PA exhibited ca. 32×MIC₀. These mutants were subsequently treated with Bi₂O₃ NPs for another 30 days and the MIC values determined (FIG. 2c, Table S1).

TABLE S1
MIC of Bi₂O₃ NPs against Ag-, ciprofloxacin-,
and meropenem-resistant PA.

| Bacteria | MIC (μg/ml) |
|---|---|
| PA_{Ag} | 1.0 |
| PA_{cip} | 0.75 |
| PAₘₑᵣₒ | 1.25 |

For AgNPs and ciprofloxacin resistant mutants, the MIC for Bi₂O₃ was 0.75 μg/mL, while for the meropenem resistant mutant the MIC for Bi₂O₃ was slightly higher at 1.25 μg/mL, indicating that the additional resistance to other materials and antibiotics does not provide any significant advantage against Bi₂O₃ NPs, which remain extremely effective even after 30 days.

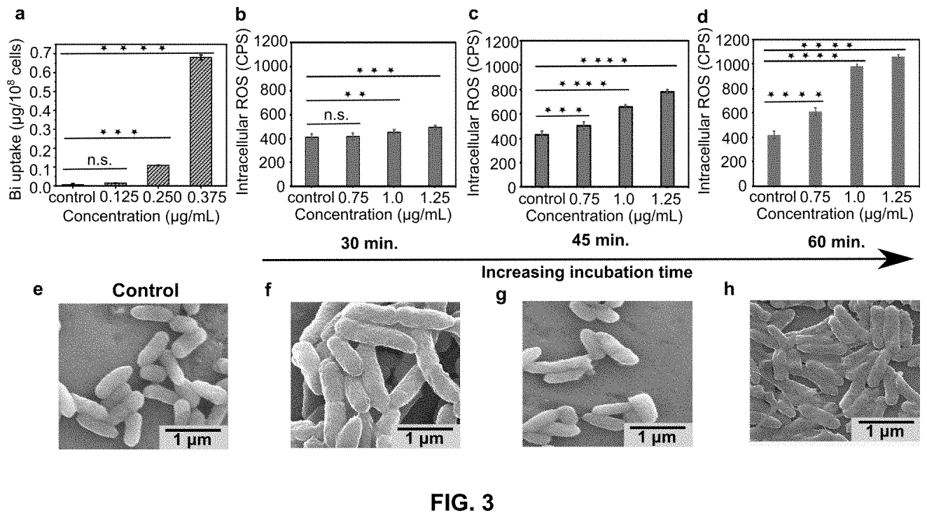

Since AgNPs-, ciprofloxacin-, and meropenem-resistance do not appear to offer any significant survival advantage for PA against Bi₂O₃, we considered that Bi₂O₃ NPs may operate by completely different mechanisms of action. Previous studies have described both reactive oxygen species (ROS) production and physical membrane disruption as possible mechanisms for nanomaterials, which are not shared by other conventional antibiotics.³¹ It is important to note that AgNPs only produce ROS indirectly through the interaction of Ag⁺ ions that are released from the oxidation of metallic Ag atoms on the surfaces of the NPs after losing the metallic bonding forces with various enzymes and iron-sulfur clusters in bacteria.^{10,32} We do not expect Bi₂O₃ NPs, however, to demonstrate an analogously significant release of Bi³⁺ ions since Bi is already in the +3 oxidized state within the crystal structure of Bi₂O₃ that has a very high lattice energy. The only possible mechanism to trigger the release of Bi³⁺ ions from the surfaces of Bi₂O₃ NPs is through acid etching, but bacteria obstinately maintain the cytoplasmic pH values above 7.³³ To better understand which of these modes of action may be operative in Bi₂O₃ NPs, we carried out several time-dependent studies. The first of which was a time-kill assay performed at various Bi₂O₃ NP concentrations (below and above the MIC) against PA (FIG. 2d). Excluding the highest concentration, which inhibited growth already in the first 6 hrs., the MIC concentration (0.75 μg/mL) and 1.3×MIC (1 μg/mL) appeared to exhibit a delayed killing effect, with the MIC being bacteriostatic and 1.3×MIC being bactericidal only after 9 hrs. To determine whether this delayed effect could be transport related, we performed Bi-uptake studies in which PA bacteria were incubated for 6 hrs. with Bi₂O₃ NPs (FIG. 3a). Following this incubation period, cells were lysed and the Bi³⁺ concentration was determined via flame atomic absorption spectroscopy (AAS). These results demonstrated that even at concentrations far below the MIC, cells take up large quantities of NPs. These results therefore indicate that the observed delay is not related to an inefficient transport or uptake inhibition.

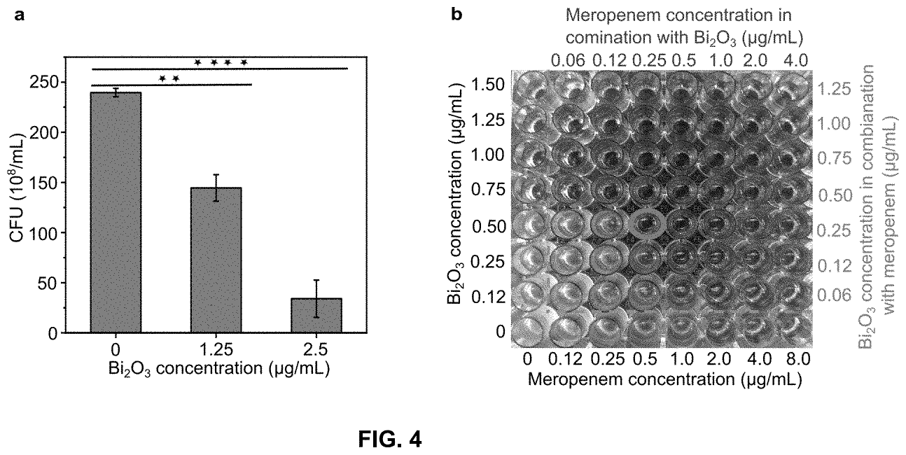

To determine whether ROS or physical membrane disruption was most likely to be the predominant mode-of-action, a side-by-side comparison of ROS generation and scanning electron microscopy (SEM) imaging was performed in PA incubated with Bi₂O₃ NPs for 30 min, 45 min, and 60 min (FIG. 3b-h). We hypothesized that if physical disruption of the membrane was occurring, then this should be visible in SEM images before significant ROS production takes place. ROS levels were quantified using a 2′,7′-dichlorofluorescein diacetate fluorescence assay. As expected, the ROS increased in both a time- and dose-dependent manner with higher Bi₂O₃ NP concentrations and longer incubation times resulting in greater levels of ROS (FIG. 3b-d). Importantly, however, ROS levels were similar to baseline control levels after 30 min, and the corresponding 30 min SEM images did not demonstrate any observable bacterial membrane damage. In fact, membrane damage could not be observed from SEM images until only after 60 min, suggesting that ROS is the primary cause for bacterial toxicity and that membrane disruption occurs as a secondary effect due to ROS-mediated damage (FIG. 3e-h). Although the exact mechanism by which ROS generation occurs is not clear, we speculate that this occurs directly as a result of a surface-based catalytic process rather than through the liberation of Bi(III) as is the case with Ag(I) from AgNPs, since Ag-resistant PA did not exhibit any significant survival advantage as compared to wild-type PA against Bi₂O₃ NPs (FIG. 4). This direct ROS generation is likely more difficult for bacteria to counter as a result of its non-specificity and numerous possible targets.

To determine whether this suspected lack of specific molecular targets within bacteria could be potentially detrimental to the use of Bi₂O₃ NPs, we measured their toxicity against human dermal fibroblasts (HDFs) and murine macrophage-like cells (RAW 264.7) to estimate the therapeutic selectivity of Bi₂O₃ NPs.

In both cases, Bi₂O₃ NPs were determined to be effectively non-toxic against both with half-maximal inhibitory concentrations (IC₅₀) of >100 μg/mL for HDF and 100 μg/mL for RAW cells (FIG. 5E). This indicates that the Bi₂O₃ NPs are highly selective for bacteria over healthy mammalian cells. This is further demonstrated by calculating the selectivity index (SI=IC₅₀/MIC) to be 145 and 133 for HDF and RAW cells against PA, respectively, which further supports the classification of Bi₂O₃ NPs as non-toxic.³⁴

Having observed both the extreme antimicrobial efficacy, the significant lack of resistance generation, and the minimal toxicity of Bi₂O₃ NPs in mammalian cells, we consider the potential use of these materials as antibacterial additives into wound dressings, implants, and catheters, which are prone in all cases to both Gram-negative and Gram-positive bacterial infections, biofilm formation, and substantial drug resistance.^{35,36} We therefore sought to determine the growth inhibitory effect of Bi₂O₃ NPs on biofilm formation as well as the ability for Bi₂O₃ NPs to act synergistically with antibiotics such as meropenem, which could potentially improve patient outcomes.

The growth inhibitory effect of Bi₂O₃ NPs on biofilm formation was determined against MRSA-derived biofilms due to their rapid growth and resilience. We observed that the growth inhibitory effect is dose-dependent and that CFUs decreased significantly going from the control to 2.5 μg/mL of Bi₂O₃ NPs, which corresponds to the MIC for MRSA (FIG. 4a). This clearly demonstrates that an alloy, polymer, or fabric containing Bi₂O₃ NPs will be strongly resistant to the formation of MRSA-biofilms. Additionally, after observing that Bi₂O₃ NPs were able to overcome mero-resistance, we checked whether Bi₂O₃ NPs may act synergistically or additively with mero since they are likely to act by independent mechanisms.³⁷

The degree of synergism was determined using a checkerboard assay in a 96-well plate (FIG. 4b). The MIC value of Bi₂O₃ NPs alone was found to be 0.75 μg/mL, and the MIC of mero alone was 2.0 μg/mL; these wells are found on the leftmost column and bottom row. Combinations of the two treatments were prepared using the 2-fold dilution method to determine the fractional inhibitory concentration (FIC; Table S2). The FIC index is used to determine whether two treatments are synergistic (χ≤0.5), additive (0.5<χ<4) or antagonistic (χ≥4).³⁸

TABLE S2
FIC index of Bi₂O₃ NPs in combination
with meropenem antibiotic.

MIC (μg/mL) against DRPA

| | Mero + | | Bi₂O₃ + | FIC |
|---|---|---|---|---|
| Mero | Bi₂O₃ | Bi₂O₃ | mero | index |
| 2.0 | 0.25 | 0.75 | 0.25 | 0.45 |

The combined treatment of meropenem with 1/3×MIC of Bi₂O₃ NPs was able to significantly reduce the MICₘₑᵣₒ from 2.0 μg/mL to 0.25 μg/mL against DRPA (FIG. 4b), and the corresponding fractional inhibitory concentration (FIC) index was found to be 0.45. This result clearly suggests that the activity of Bi₂O₃ NPs and mero are synergistic (Table S2). We expect that this synergism arises as a result of the independent mechanisms of action of Bi₂O₃ NPs and mero, in agreement with the observed ability of Bi₂O₃ NPs to treat mero-resistant PA mutants.

The potent broad-spectrum antimicrobial activity of Bi₂O₃ NPs indicates that this nanomaterial may have potential as an alternative to both mupirocin and fusidic acid for treating skin and soft tissue infections (SSTIs) by MRSA.

As an opportunistic pathogen, Staphylococcus aureus (SA) is the most common cause of skin and soft tissue infections (SSTIs) because about 30% of people are colonized by SA on the skin and in the nares. With the emergence of methicillin-resistant Staphylococcus aureus (MRSA), treatment of SSTIS by MRSA has become increasingly problematic. If not treated in a timely manner, patients of SSTIs by MRSA may develop more serious and life-threatening systemic infections.

Currently, there are two topical antibiotics that are still effective against MRSA. This first one is mupirocin (Bactroban®) and the second one is fusidic acid or fusidate. In light of rising antimicrobial resistance (AMR), an increasing number of MRSA strains are found to be resistant to mupirocin due in large part to its widespread and routine use in the community and hospitals for nasal SA decolonization. The situation of fusidate resistance found in MRSA strains is even worse. As the development of resistance to fusidate involves a single point mutation, the generic barriers to mutation are hence low, diminishing the efficacy of fusidate as a topical monotherapy for treating SSTIs by MRSA. As the result, fusidate has never been approved for use as a topical antibiotic in the US but remains common in Europe. It should be noted that both mupirocin and fusidate are prescription-only medications, which excludes some patients from seeking treatment for their seemingly harmless SSTIs by MRSA.

To test whether topical creams of Bi₂O₃ NPs can be used in place of mupirocin or fusidate as an over-the-counter medication to treat SSTIs by MRSA, three polyethylene glycol (PEG) based ointments containing 1%, 2% and 4% Bi₂O₃ NPs were prepared, and the antimicrobial activity of these topical creams were studied.

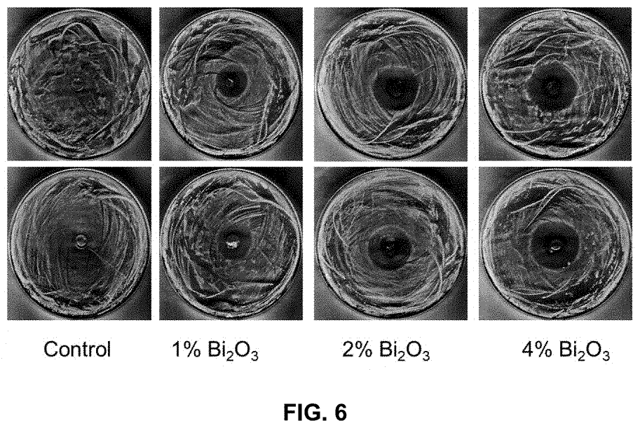

As shown in FIG. 6, treatment with the topical creams containing 1%, 2% and 4% Bi₂O₃ NPs in the wildtype MRSA bacteria resulted in a progressive increase in the zone of inhibition in comparison to the treatment with PEG as the vehicle control, indicating that similar to mupirocin and fusidate, the Bi₂O₃ NPs can be delivered topically to treat SSTIs by MRSA.

Accordingly, in one embodiment of the present invention, topical creams comprising the Bi₂O₃ NPs and a carrier are provided, wherein the Bi₂O₃ NPs are present in an amount from about 0.1 to about 25 wt. %, desirably in an amount from about 0.25 to about 15 wt. %, and preferably from about 0.5 to about 10 wt. % based on the total weight of the composition. Polyethylene glycol is the preferred carrier in one embodiment.

In summary, we have demonstrated that Bi₂O₃ NPs are not only extremely potent broad-spectrum antimicrobial materials with MIC values below those of AgNPs, but that they also maintain this efficacy against Ag-resistant, cipro-resistant, and mero-resistant mutants of PA bacteria. Furthermore, we have demonstrated for the first time that Bi₂O₃ NPs significantly inhibit the generation of new resistant phenotypes even after 30 passages. These results suggest that metal-oxide semiconductors NPs or quantum dots may in fact be a very small solution to the very large problem of antimicrobial resistance.

### EXPERIMENTAL SECTION

Chemical reagents and biological material. All chemical reagents were obtained from commercial sources and used without any further purification. Bismuth (III) pentahydrate (98%), nitric acid (65%), poly(vinylpyrrolidone) (Mw=8000), dimethyl sulfoxide (≥9.5%), silver nitrate (≥99%), sodium hydroxide, D-maltose, ciprofloxacin (≥98%), and meropenem (≥98%) were purchased from Sigma Aldrich. Ammonium hydroxide (28-30%) was purchased from Acros organics. Bacterial strains, growth media and antibiotics. Gram-positive bacteria (SA; ATCC 6538, MRSA; ATCC BAA-44) and Gram-negative bacteria (PA; ATCC 15692, DRPA; ATCC BAA-2108) were purchased from American Type Culture Collection. Tryptic broth powder (TSB), tryptic soy agar (TSA), nutrient broth (NB) and nutrient agar (NA) were purchased from Fisher Scientific.

### Synthetic Methods

Synthesis of Bi₂O₃: An aqueous solution of Bi(NO₃)₃ (8.25 mM) was prepared under acidic conditions (20 mL HNO₃). To this, 40 mL of an aqueous ammonia solution were added dropwise under constant stirring. As the ammonia solution was added, a white precipitate of, most likely Bi(OH)₃, was obtained. This product was filtered and washed three times with deionized water (DI) water, at which point the pH of the washing was ˜7.0. This white product was then dried on a hotplate at 100° C. for 2 hrs and then finely ground before being calcined at 525° C. for 4 hrs to yield bulk Bi₂O₃, which was pale-yellow in color.

Synthesis of Bi₂O₃ NPs: Bi₂O₃ NPs were prepared sonomechanically from the previously prepared bulk Bi₂O₃. To do so, 1 mg of finely ground bulk Bi₂O₃ was dispersed in 1 mL of DMSO containing 100 mg of PVP-8k. This mixture was then sonicated for 18 hrs.

Synthesis of Ag NPs: Silver nanoparticles were synthesized by a modified Tollens method, by reducing [Ag(NH₃)₂]⁺ with D-maltose.⁹

Characterization

Powder XRD: Powder patterns were obtained using a Rigaku MiniFlex 600 X-ray diffractometer using Cu Kα radiation, a Kβ-filter, a LynxEye PSD detector, and an incident beam Ge 111 monochromator. Patterns were measured from 10 to 80° 28 with a step-size of 0.01446° and an exposure time of 800 sec. Rietveld refinements were performed using GSAS II.³⁹

TEM and HRTEM: TEM grids were prepared in the following manner. Bi₂O₃ NPs were first dispersed in ethanol and sonicated for 30 minutes. Then, a droplet of the suspension was placed onto a carbon-coated copper TEM grid (400-mesh) and the samples were allowed to air-dry before the analysis. TEM images obtained using a FEI Tecnai F20 (200 kV) equipped with a field emission gun and an integrated scanning TEM (STEM) unit. TEM images were processed using FIJI.⁴⁰

SEM imaging of bacteria. The morphology of bacterial samples was characterized using a Quanta 450 SEM operating at 15 kV accelerating voltage. Typically, desired bacteria (1×10⁹ CFU/mL) were treated with the nanoparticles at different concentrations for different time periods and then bacterial suspensions were centrifuged at 3750 rpm for 7 minutes at 4° C. Later on, bacterial pellets were resuspended into 1 mL of PBS twice. Subsequently, the bacteria were fixed with PBS containing 2.5% glutaraldehyde. Again, pellets were washed with PBS three times and were subjected to 1% tannic acid. After that, samples were dehydrated with a series of graded ethanol solutions, dried in air, and coated with gold. Finally, the SEM images were taken.

Biological Assays

Preparation of Test Solutions. Test solutions were prepared by dissolving the desired amount of Bi₂O₃ NPs and PVP (by weight) in DMSO. Nutritious mediums for microorganisms (TSB, TSA, NB and NA) were prepared from the powder by dissolving the desired amount in DI water.

Bacteria Suspensions. To culture the bacterial suspensions, an isolated colony of Gram-positive bacteria was added to 5 mL of TSB media and an isolated colony of Gram-negative bacteria was added to 5 mL of NB media followed by incubation for 18 hrs. at 37° C. Bacterial cell density was determined by optical density (OD) measurements 600 nm using a SpectraMax M4 Microplate Reader.

M/C assay. NP solutions with different concentrations were diluted in TSB and inoculated with a bacterial strain at a concentration of 10⁶ CFU/mL in a 96 well-plate followed by incubating the bacteria at 37° C. for 18 hours. After that, the MIC of NPs were determined as the lowest concentration that inhibits visible growth of the tested microorganisms with unaided eyes and OD reading at 600 nm using a microtiter plate reader.

Colony Forming Unit (CFU/ml) Assays. A control of bacteria without nanoparticles in TSB media were used in every cell culture study. After 18 hrs. of incubation with the nanoparticles, a diluted suspension was spread on agar plates using glass spreaders. After 18 hrs. of incubation at 37° C., the number of colonies were counted in each plate and converted into CFU/mL values. All measurements were performed in biological and technical triplicates. The MIC was determined as the lowest concentration of a drug that could inhibit the growth of a microorganism by both visual reading and OD at 600 nm using a microtiter plate reader.

Bi uptake by PA measured with AAS. The cellular uptake of Bismuth in PA was determined using a flame AAS (Buck Scientific Atomic Absorption Spectrometer, Model 210 VGP). To prepare the calibration curve, a range of diluted solutions were prepared from a commercially available standard solution (1000 ppm). The hollow cathode lamp was used to analyze metal concentrations that operated at 10 mA and an air acetylene flame was used for all the measurements. Bi₂O₃ concentrations of 0.125 μg/mL, 0.25 μg/mL, and 0.375 μg/mL were used. After 6 hrs. of incubation at 37° C., a 500 μL aliquot of the bacterial suspension was removed, and the number of CFU was determined by agar plate method. The remaining bacterial suspensions were centrifuged at 25° C. and 3700 rpm for 7 min. The supernatant was discarded, and the bacterial pellet was washed three times with DI water. This pellet was then digested using 70% HNO₃ to destroy the organic material; the metal ions in these solutions were then converted to oxides by calcination of the samples at 620° C. for 5 hrs. These metal oxides were then dissolved in aqua regia, and the bismuth concentrations were determined.

Quantification of reactive oxygen species. The generation of intracellular ROS by Bi₂O₃ NPs in PA was determined using a DCFH-DA assay. First, 1 mL of overnight cultured PA was collected by centrifugation (3750 rpm, 7 min.) and resuspended in 400 μL of fresh NB medium. Then, the bacterial cells were incubated with different concentrations of Bi₂O₃ compared with a control for 30 min, 45 min and 60 min separately. Next, the cells of each group were harvested again by centrifugation and washed twice with PBS. The bacterial cells were incubated with 500 μL of 20 μM DCFH-DA dye in PBS at 37° C. while shaking for 30 min. The intracellular ROS level was examined by fluorescence microscopy with the excitation and emission wavelengths set at 497 nm and 529 nm, respectively.

Drug resistance study. The MIC was determined using a broth dilution method. Bacteria (˜1.0×10⁶ CFU/ml) were cultured in a NB medium with either Bi₂O₃ NPs, ciprofloxacin, AgNPs or meropenem. The as-prepared bacterial solutions were incubated at 37° C. for 24 h. After incubation, the MIC was determined as the lowest concentration that inhibited visible growth of the tested microorganisms with unaided eyes. This same procedure was done by serial passaging of the bacteria until 30 days. Later on, three types of mutant DSPA were prepared by treating with Ag NPs, ciprofloxacin and meropenem for 15 days. At this point, the drug-resistant bacterial mutants were taken and subsequently treated with Bi₂O₃ NPs for another 30 days.

Biofilm inhibition assay. An overnight culture of MRSA (ATCC BAA-44) was diluted to a final cell concentration of 1×10⁶ CFU/mL and transferred (100 μL) to a 96-well plate containing Bi₂O₃ NPs at varying concentrations ranging from 1.25 to 5 μg/mL. The bacterial cells were incubated with stationary phase for 24 hours at 37° C. to form biofilms. After incubation, the biofilm was gently washed to keep the biofilms intact and resuspended with PBS. For the quantification of the number of viable bacteria in the biofilm, the biofilms were gently destroyed and plated on TSA after serial dilutions of each suspension. The number of viable bacteria in the sample was obtained using agar plate counting method as described above and results were expressed as CFU changes with respect to the control (without Bi₂O₃ NPs).

Mammalian cell viability assay. Cytotoxicity of Bi₂O₃ NPs toward mammalian cells was determined using an MTT viability assay. Mammalian cells (RAW 264.7 cells and normal human dermal fibroblasts) were seeded in a 96-well plate at a density of 4×10⁴ cells per well with a DMEM high-glucose medium and incubated for 24 hours at 37° C. in an atmosphere of 5% CO₂ and 95% air. Cells in each well were then treated with 100 μL of fresh medium containing the various test concentrations of Bi₂O₃ NPs and then incubated for 24 hours. After the cells were incubated with 10 μL of the MTT reagent for 2 hours at 37° C., 100 μL of detergent reagent was added to all wells and the plate was left, covered, in the dark for 2 hours at 37° C. The absorbance was measured at 570 nm using a microplate reader (SpectraMax M4). The assay was run in triplicate, and the results were presented as percentage of viable cells with respect to the viability of untreated control cells.

Checkerboard assay. The checkerboard assay was performed in a 96-well plate as previously described.³⁷ The antibiotic meropenem was 2-fold serially diluted along the row-axis, while Bi₂O₃ NPs was 2-fold serially diluted along the column-axis to create a matrix in which each well consists of a combination of both Bi₂O₃ NPs and meropenem at different concentrations. Each well was inoculated with DRPA (CC BAA-2108) to yield approximately 1×10⁶ https.//www.sciencedirect.com/topics/immunology-and-mtcrobioiogy/colony-forming-unit CFU/mL in a 100 -μL final volume, incubated for 18 hours at 37° C. and examined for visibility to determine the MIC. The FIC of Bi₂O₃ NPs is calculated by dividing the MIC of Bi₂O₃ NPs in the presence of the antibiotic by the MIC of Bi₂O₃ NPs in the absence of the antibiotic. Similarly, the FIC of a given antibiotic was calculated by dividing the MIC of the antibiotic in the presence of Bi₂O₃ NPs by the MIC of the antibiotic the absence of 1 Bi₂O₃ NPs. The FIC index, obtained by summating both FIC values, can then be interpreted as synergistic (χ≤0.5), additive (0.5<χ<4), or antagonistic (χ≥4).

Statistical analysis. Statistical analysis was performed using GraphPad Prism version 8.0 software. A two-tailed unpaired t-test was used to determine statistical significance between two groups. A statistical significance among multiple groups was analyzed using One-way ANOVA followed by Holm-Sidak comparisons test. For all analyses, p-value of less than 0.05 was considered to be statistically significant. Data were presented as mean±standard deviation (mean±s.d). The in vitro studies were run with at least three biological replicates and each biological replicate has three technical replicates.

### REFERENCES

1 Antibiotic Resistance, <https://www.who.int/news-room/fact-sheets/detail/antibiotic-resistance> (2020).
2 Nelson, R. E. et al. National Estimates of Healthcare Costs Associated With Multidrug-Resistant Bacterial Infections Among Hospitalized Patients in the United States. Clin Infect Dis 72, S17-S26, doi:10.1093/cid/ciaa1581 (2021).
3 Blaskovich, M. A. T. The Fight Against Antimicrobial Resistance Is Confounded by a Global Increase in Antibiotic Usage. ACS Infect. Dis. 4, 868-870, doi:10.1021/acsinfecdis.8b00109 (2018).
4 Blair, J. M., Webber, M. A., Bayley, A. J., Ogbolu, D. O. & Piddock, L. J. Molecular mechanisms of antibiotic resistance. Nat. Rev. Microbiol. 13, 42-51, doi:10.1038/nrmicro3380 (2015).
5 Zhang, G. F., Liu, X., Zhang, S., Pan, B. & Liu, M. L. Ciprofloxacin derivatives and their antibacterial activities. Eur. J. Med. Chem. 146, 599-612, doi:10.1016/j.ejmech.2018.01.078 (2018).
6 Nägeli, C. V. Über oligodynamische Erscheinungen in lebenden Zellen. Neue Denkschriften der Allg. Schweizerischen Gesellschaft für die Gesammten Naturwissenschaften 33 (1893).
7 Lemire, J. A., Harrison, J. J. & Turner, R. J. Antimicrobial activity of metals: mechanisms, molecular targets and applications. Nat. Rev. Microbiol. 11, 371 384, doi:10.1038/nrmicro3028 (2013).
8 Roman, M. et al. Spatiotemporal distribution and speciation of silver nanoparticles in the healing wound. Analyst 145, 6456.6469, doi:10.1039/d0an00607f (2020).
9 Panacek, A. et al. Bacterial resistance to silver nanoparticles and how to overcome it. Nat Nanotechnol 13, 65-71, doi:10.1038/s41565-017-0013-y (2018).
10 Medici, S., Peana, M., Nurchi, V. M. & Zoroddu, M. A. Medical Uses of Silver: History, Myths, and Scientific Evidence. J. Med. Chem. 62, 5923-5943, doi:10.1021/acs.jmedchem.8b01439 (2019).
11 Ayala-Nunez, N. V., Lara Villegas, H. H., del Carmen Ixtepan Turrent, L. & Rodriguez Padilla, C. Silver Nanoparticles Toxicity and Bactericidal Effect Against Methicillin-Resistant Staphylococcus aureus: Nanoscale Does Matter. NanoBiotechnology 5, 2-9, doi: 10.1007/s12030-009-9029-1 (2009).
12 Gunawan, C. et al. Widespread and Indiscriminate Nanosilver Use: Genuine Potential for Microbial Resistance. ACS Nano 11, 3438-3445, doi:10.1021/acsnano.7b01166 (2017).
13 Kumar, A., Vemula, P. K., Ajayan, P. M. & John, G. Silver-nanoparticle-embedded antimicrobial paints based on vegetable oil. Nat Mater 7, 236-241, doi: 10.1038/nmat2099 (2008).
14 Nesporova, K. et al. Effects of wound dressings containing silver on skin and immune cells. Sci Rep 10, 15216, doi:10.1038/s41598-020-72249-3 (2020).
15 Kalantari, K. et al. Wound dressings functionalized with silver nanoparticles: promises and pitfalls. Nanoscale 12, 2268-2291, doi:10.1039/c9nr08234d (2020).
16 Bianchini, A., Playle, R. C., Wood, C. M. & Walsh, P. J. Mechanism of acute silver toxicity in marine invertebrates. Aquat Toxicol 72, 67-82, doi: 10.1016/j.aquatox.2004.11.012 (2005).
17 Choi, O. et al. The inhibitory effects of silver nanoparticles, silver ions, and silver chloride colloids on microbial growth. Water Res 42, 3066-3074, doi: 10.1016/j.watres.2008.02.021 (2008).
18 Wang, R., Li, H., Ip, T. K.-Y. & Sun, H. in Medicinal Chemistry Advances in Inorganic Chemistry 183-205 (2020).
19 Duffin, R. N., Werrett, M. V. & Andrews, P. C. in Medicinal Chemistry Advances in Inorganic Chemistry 207-255 (2020).
20 Yuan, S. et al. Metallodrug ranitidine bismuth citrate suppresses SARS-CoV-2 replication and relieves virus-associated pneumonia in Syrian hamsters. Nat Microbiol 5, 1439-1448, doi: 10.1038/s41564-020-00802-x (2020).
21 Chattopadhyay, A. et al. An Inexpensive Bismuth-Petrolatum Dressing for Treatment of Burns. Plast Reconstr Surg Glob Open 4, e737, doi: 10.1097/GOX.0000000000000741 (2016).
22 Santos, R. M., Sampaio, C. P., Moraes, D. P. & Lima, R. L. Evaluation of the Effects of Bismuth Subgallate on Wound Healing in Rats. Histological Findings. Int Arch Otorhinolaryngol 20, 377-381, doi:10.10551s-0036-1583760 (2016).
23 Wang, R. et al. Bismuth antimicrobial drugs serve as broad-spectrum metallo-beta-lactamase inhibitors. Nat. Commun. 9, 439, doi:10.1038/s41467-018-02828-6 (2018).
24 Li, H., Wang, R. & Sun, H. Systems Approaches for Unveiling the Mechanism of Action of Bismuth Drugs: New Medicinal Applications beyond Helicobacter Pylori Infection. Acc Chem Res 52, 216-227, doi:10.1021/acs.accounts.8b00439 (2019).
25 Stephens, L. J. et al. Is Bismuth Really the “Green” Metal? Exploring the Antimicrobial Activity and Cytotoxicity of Organobismuth Thiolate Complexes. Inorg. Chem. 59, 3494-3508, doi:10.1021/acs.inorgchem.9b03550 (2020).
26 Shahbazi, M. A. et al. The versatile biomedical applications of bismuth-based nanoparticles and composites: therapeutic, diagnostic, biosensing, and regenerative properties. Chem Soc Rev 49, 1253-1321, doi:10.1039/c9cs00283a (2020).
27 Katoch, V. et al. Microflow synthesis and enhanced photocatalytic dye degradation performance of antibacterial Bi2O3 nanoparticles. Environ Sci Pollut Res Int 28, 19155-19165, doi:10.1007/s11356-020-11711-1 (2021).
28 Pendleton, J. N., Gorman, S. P. & Gilmore, B. F. Clinical relevance of the ESKAPE pathogens. Expert Review of Anti-infective Therapy 11, 297-308, doi:10.1586/eri.13.12 (2013).
29 Magill, S. S. et al. Multistate point-prevalence survey of health care-associated infections. N. Engl. J. Med. 370, 1198-1208, doi:10.1056/NEJMoa1306801 (2014).
30 CDC. ANTIBIOTIC RESISTANCE THREATS IN THE UNITED STATES. U.S. Department of Health and Human Services, doi: 10.15620/cdc:82532 (2019).
31 Tu, Y. et al. Destructive extraction of phospholipids from Escherichia coli membranes by graphene nanosheets. Nat Nanotechnol 8, 594-601, doi: 10.1038/nnano.2013.125 (2013).
32 Betts, H. D., Whitehead, C. & Harris, H. H. Silver in biology and medicine: opportunities for metallomics researchers. Metallomics 13, doi:10.1093/mtomcs/mfaa001 (2021).
33 Krulwich, T. A., Sachs, G. & Padan, E. Molecular aspects of bacterial pH sensing and homeostasis. Nature reviews. Microbiology 9, 330-343, doi:10.1038/nrmicro2549 (2011).
34 Cagno, V. et al. Broad-spectrum non-toxic antiviral nanoparticles with a virucidal inhibition mechanism. Nat Mater 17, 195-203, doi:10.1038/nmat5053 (2018).
35 Olejnickova, K., Hola, V. & Ruzicka, F. Catheter-related infections caused by Pseudomonas aeruginosa: virulence factors involved and their relationships. Pathog Dis 72, 87-94, doi:10.1111/2049-032X.12188 (2014).
36 Jacobsen, S. M., Stickler, D. J., Mobley, H. L. & Shirtliff, M. E. Complicated catheter-associated urinary tract infections due to Escherichia coli and Proteus mirabilis. Clin. Microbiol. Rev. 21, 26-59, doi:10.1128/CMR.00019-07 (2008).
37 Meletiadis, J., Poumaras, S., Roilides, E. & Walsh, T. J. Defining fractional inhibitory concentration index cutoffs for additive interactions based on self-drug additive combinations, Monte Carlo simulation analysis, and in vitro-in vivo correlation data for antifungal drug combinations against Aspergillus fumigatus. Antimicrob Agents Chemother 54, 602-609, doi:10.1128/AAC.00999-09 (2010).
38 Meletiadis, J., Poumaras, S., Roilides, E. & Walsh, T. J. Defining fractional inhibitory concentration index cutoffs for additive interactions based on self-drug additive combinations, Monte Carlo simulation analysis, and in vitro-in vivo correlation data for antifungal drug combinations against Aspergillus fumigatus. Antimicrobial agents and chemotherapy 54, 602-609 (2010).
39 Toby, B. H. & Von Dreele, R. B. GSAS-II: the genesis of a modern open-source all purpose crystallography software package. J. Appl. Crystallogr. 46, 544-549, doi:doi:10.1107/S0021889813003531 (2013).
Schindelin, J. et al. Fiji: an open-source platform for biological-image analysis. Nature Methods 9, 676-682, doi:10.1038/nmeth.2019 (2012).

What is claimed is:

1. An antimicrobial composition, comprising:
α-Bi₂O₃ nanoparticles prepared by ultrasonication in a solvent in the presence of a water-soluble and biocompatible polymer, wherein the α-Bi₂O₃ nanoparticles have an average size of from about 2 to about 50 nm and are surface-coated with the water-soluble and biocompatible polymer.

2. The antimicrobial composition according to claim 1, wherein said α-Bi₂O₃ nanoparticles are capable of eradicating antibiotic-susceptible Gram-negative (MIC=0.75 μg/mL vs. P. aeruginosa) and Gram-positive (MIC=2.5 μg/mL vs. S. aureus) bacteria.

3. The antimicrobial composition according to claim 1, wherein said α-Bi₂O₃ nanoparticles are capable of eradicating Ag-resistant Gram-negative (MIC=1.0 μg/mL) and Gram-negative carbapenem-resistant strains (MIC=1.25 μg/mL) of bacteria.

4. The antimicrobial composition according to claim 1, wherein said α-Bi₂O₃ nanoparticles are capable of sensitizing meropenem in Gram-negative bacteria with the FIC index of 0.45.

5. The antimicrobial composition according to claim 1, wherein the Bi₂O₃ nanoparticles have an average size of about 4 to about 30 nm.

6. The antimicrobial composition according to claim 5, wherein the Bi₂O₃ nanoparticles have an average size of 6.03+/−0.93 nm.

7. The antimicrobial composition according to claim 1, wherein the composition comprises a carrier.

8. The antimicrobial composition according to claim 7, wherein the carrier comprises polyethylene glycol.

9. The antimicrobial composition according to claim 5, wherein the Bi₂O₃ are present in an amount from about 0.1 to about 25 wt. % based on the total weight of the composition.

10. The antimicrobial composition according to claim 5, wherein the Bi₂O₃ are present in an amount from about 0.25 to about 15 wt. % based on the total weight of the composition.

11. The antimicrobial composition according to claim 6, wherein the Bi₂O₃ are present in an amount from about 0.5 to about 10 wt. % based on the total weight of the composition.

12. The antimicrobial composition according to claim 1, wherein the water-soluble and biocompatible polymer is one or more of polyvinylpyrrolidone, polyethylene glycol, polyacrylamide, polyacrylic acid, and a polyvinyl alcohol.

13. The antimicrobial composition according to claim 12, wherein the water-soluble and biocompatible polymer consists of polyvinylpyrrolidone.

14. A method for preparing the antimicrobial composition according to claim 1 comprising the steps of:
sonicating bulk Bi₂O₃ powder in the presence of the water-soluble and biocompatible polymer and a solvent; and
recovering the sonicated Bi₂O₃.

15. The method according to claim 14, further including the step of combining a carrier with the Bi₂O₃.

16. The method according to claim 15, wherein the carrier is polyethylene glycol.

17. The method according to claim 14, wherein the solvent is one or more of methanol, ethanol, N,N′-dimethyformamide (DMF), acetonitrile, dimethyl sulfoxide (DMSO) and water.

18. The method according to claim 14, wherein the water-soluble and biocompatible polymer is one or more of polyvinylpyrrolidone, polyethylene glycol, polyacrylamide, polyacrylic acid, and polyvinyl alcohol.

19. The method according to claim 18, wherein the water-soluble and biocompatible polymer is polyvinylpyrrolidone.
